(19) United States
(12) Patent Application Publication
(10) Pub. No.: US 2016/0068798 A1
(43) Pub. Date: Mar. 10, 2016

(54) SYSTEMS AND METHODS FOR SINGLE CELL ISOLATION AND ANALYSIS

(71) Applicant: THE REGENTS OF THE UNIVERSITY OF MICHIGAN, ANN ARBOR, MI (US)

(72) Inventors: Euisik Yoon, Superior Township, MI (US); Yu-Chih Chen, Ann Arbor, MI (US)

(21) Appl. No.: 14/848,834

(22) Filed: Sep. 9, 2015

Related U.S. Application Data
(60) Provisional application No. 62/047,823, filed on Sep. 9, 2014.

(51) Int. Cl. C12M 1/00; C12Q 1/24; C12M 3/06

(52) U.S. Cl. CPC C12M 47/04; C12M 23/16; C12Q 1/24

(57) ABSTRACT

The present disclosure relates to devices, systems, and methods for single cell isolation and analysis. In particular, the present disclosure relates to laser detachment systems and methods for isolating single cells suitable for culture and molecular analysis.

The present application claims priority to U.S. Provisional Patent Application Ser. No. 62/047,823, filed Sep. 9, 2014, the disclosure of which is herein incorporated by reference in its entirety.

### FIELD OF THE INVENTION

The present disclosure relates to devices, systems, and methods for single cell isolation and analysis. In particular, the present disclosure relates to laser detachment systems and methods for isolating single cells suitable for culture and molecular analysis.

### BACKGROUND OF THE INVENTION

Cancer cell heterogeneity is one of key challenges in modern cancer study. Due to the genomic instability of cancer cells (Negrini et al., (2010) Nat Rev Mol Cell Biol. 11(3):220-228), certain cells may have higher capability of drug resistance, metastasis and tumorgenesis (Visvader and Lindeman G J (2008) Nat Rev Cancer. 8(10):755-768). Studying these sub-populations separately can lead to effective therapeutic targets. For example, the state transition, such as the epithelial-to-mesenchymal transition (EMT) is one of key events in the tumor development and metastasis (Yang and Weinberg R A. (2008) Dev Cell. 14(6):818-29).

These mechanisms cannot be easily studied by conventional dish-based assays. Recent development of microfluidics has provided single-cell assay capability by isolating and culturing cells in an array of microchambers (Chung et al., Appl. Phys. Lett, 98(12), 3701 (2011)). However, these methods lack the ability to retrieve a target single cell for further analysis (e.g., genotyping) and assays (e.g., drug-screening).

Conventional cell detachment schemes, such as trypsinization or PNIPAAm-based detachment (Canavan et al, (2005) J Biomed Mater Res A. 75(1):1-13) do not provide any spatial resolution; they give blank detachment of entire cells from the substrate. The PALM CombiSystem developed by Zeiss can detach cells adhered on a laser absorbing film. However, detaching cells from the special film limits spatial resolution and it is difficult to handle cells over the film. On top of that, the cell detachment based on photodegradation of the substrate film, which generates acid, may lead to toxicity to the cells (Kimio et al., Proceedings of MicroTAS 2013 100-102).

Recently, an IR-triggered detachment method of single cells on CNT substrates was reported (Sada et al, (2011) ACS Nano. 5(6):4414-21). However, cell viability was poor because of heat-induced cell necrosis under direct laser irradiation. Recently, cell detachment using ultrasound-induced cavitation was demonstrated (Baac et al., (2012) Sci. Rep. 2, 989), but unfortunately this approach only works on Petri dishes and is not compatible with microfluidic arrangement due to acoustic attenuation by PDMS.

### SUMMARY OF THE INVENTION

The present disclosure relates to devices, systems, and methods for single cell isolation and analysis. In particular, the present disclosure relates to laser detachment systems and methods for isolating single cells suitable for culture and molecular analysis.

The devices, system, kits, and methods of embodiments of the present disclosure provide the advantage of removing single cells from a colony of cells such that the cells are suitable for down-stream analysis (e.g., further culture or molecular analysis).

For example, in some embodiments, the present disclosure provides a kit or system, comprising: a) a multi-layer substrate comprising a light absorbing layer and a polymer layer; and b) a source of optical or electrical energy. In some embodiments, the substrate comprises a plurality of bubbles. In some embodiments, the polymer layer is, for example, polydimethylsiloxane (PDMS), polymethyl methacrylate (PMMA), or polystyrene (PS). In some embodiments, the polymer layer is approximately 1-100 μm thick. In some embodiments, the light absorbing layer is a carbon nanotube film or a metal film. In some embodiments, the light absorbing layer is chemical vapor deposited. In some embodiments, the light absorbing layer is patterned. In some embodiments, the polymer layer is on top of the light absorbing layer. In some embodiments, the polymer film has a plurality of cells attached thereof. In some embodiments, the cells are growing. In some embodiments, contacting the substrate with the optical energy source generates heat that leads to the expansion of said bubbles or deformation of the substrate polymer and detachment of cells growing on said bubbles. In some embodiments, the system further comprises a plurality of reagents and devices for performing analysis of the cells. In some embodiments, the source of optical energy is a pulsed laser (e.g., a nanosecond pulsed laser). In some embodiments, the substrate is a component of a microfluidic chamber. In some embodiments, the microfluidic chamber comprises a fluid exchange system. In some embodiments, the microfluidic chamber comprises a plurality of chambers, wherein each of the chambers is configured to hold a single cell or cell colony.

In further embodiment, the present disclosure provides a method, comprising: contacting a multi-layer substrate comprising a light absorbing layer and a polymer layer wherein said polymer film has a plurality of cells attached thereof with a source of optical energy such that one or more single cells are detached from said substrate to generate detached cells and attached cells. In some embodiments, the method further comprises the step of performing molecular analysis on the detached cell and/or said attached cells. In some embodiments, the molecular analysis is, for example, gene expression analysis, nucleic acid methylation analysis, gene copy number variation analysis, sequencing analysis, protein analysis, or mutation analysis. In some embodiments, the method is in vivo (e.g., devices reside in vivo). In some embodiments, cells are isolated.

Additional embodiments will be apparent to persons skilled in the relevant art based on the teachings contained herein.

### DESCRIPTION OF THE DRAWINGS

FIG. 1 shows a schematic diagram of single cell detachment setup.

FIG. 2 shows a schematic diagram of a single cell assay platform: (a) cross-sectional view and (b) 3D micro-chamber schematics with a captured cell.

FIG. 3 shows SEM images of the substrates: (a) the CVD-grown CNT forest on quartz substrate (b) the embedded CNTs in the PDMS layer after spin coating of PDMS.

FIG. 4 shows the process of single cell detachment: (a) before detachment, (b) after detachment, (c) the detached cell flowing away.

FIG. 5 shows partial cell detachment: (a) before detachment, (b) after detachment, (c) partially detached cell anchored on one side and detached on the other.

FIG. 6 shows exemplary cell retrieval process: (a) Cell loading phase—The media flows downward in all chambers and cells are hydro-dynamically captured in each chamber, (b) Cell harvesting to the left—After cell detachment, cells are harvested in the lower chambers (the second row) first by applying pressure from right to left. The detached cells in the lower chambers are guided upward and then collected in the left outlet (c) Cell harvesting to the right—Cells are retrieved in the upper chamber (the first row) by applying pressure from left to right. The detached cells in the upper chambers are guided upward and then collected in the right outlet. Using the alternating parallel channels in an array, cells are retreived from all the chambers.

FIG. 7 shows the process of sequentially detachment of 4 cells in a chamber: (a) before detachment, there were 3 ALDH+ cells and 1 ALDH− cell (b) the ALDH− cell was detached and harvested first, (c) the second cell was detached and harvested, (d) the third cell was detached and harvested, and (e) the last cell was detached and harvested.

FIG. 8 shows the recovery of a skov3 cell after laser detachment: (a) before detachment, (b) after detachment, (c) 1 day recovery in the same chamber, (d) 7 days recovery showing healthy proliferation.

FIG. 9 shows quantitative recovery data of skov3 cells 2 days after the detachment by laser and by trypsin, respectively. Laser detachment showed a higher percentage of re-adhered and proliferated cells.

### DEFINITIONS

To facilitate an understanding of the present invention, a number of terms and phrases are defined below:

As used herein, the term “optical energy source” refers to any source of optical (e.g., light on the visible or non-visible spectrum) energy that generates heat (e.g., when contacted with a “light absorbing layer”). In some embodiments, optical energy is a laser. In some embodiments, optical energy is delivered in short pulses (e.g., by a nanosecond pulsed laser or other pulsed laser).

As used herein, the term “electrical energy source” refers to any source of electrical energy (e.g., electricity) that generates heat (e.g., when contacted with a “light absorbing layer”).

As used herein, the term “light absorbing layer” refers to a thin film or layer of a material that absorbs optical energy and generates heat when contacted with a source of optical (or electrical) energy. Examples include, but are not limited to, carbon nanotubes and metals (e.g., metals with conductive properties). Examples of suitable metals for use in or as “light absorbing layers” include, but are not limited to, Pd/Au alloys or Au alone.

As used herein, the term “carbon nanotube” or “CNT” refers to allotropes of carbon with a cylindrical nanostructure. Nanotubes are members of the fullerene structural family. Their name is derived from their long, hollow structure with the walls formed by one-atom-thick sheets of carbon, called graphene. These sheets are rolled at specific and discrete (“chiral”) angles, and the combination of the rolling angle and radius decides the nanotube properties; for example, whether the individual nanotube shell is a metal or semiconductor. Nanotubes are categorized as single-walled nanotubes (SWNTs) and multi-walled nanotubes (MWNTs). Individual nanotubes naturally align themselves into “ropes” held together by van der Waals forces, more specifically, pi-stacking.

As used herein, the term “bubble” refers to an air or gas filled cavity in-between multiple layers of solid material. In some embodiments, bubbles are located between multiple layers of the substrates described herein. In some embodiments, bubbles are formed by energy that is absorbed by one or more of the layers that results in the generation of heart.

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE INVENTION

The present disclosure relates to devices, systems, and methods for single cell isolation and analysis. In particular, the present disclosure relates to laser detachment systems and methods for isolating single cells suitable for culture and molecular analysis.

Experiments conducted during the course of development of embodiments of the present disclosure provide systems and methods for retrieving viable cells at single cell resolution from the microfluidic chip with unprecedented spatial resolution. Pulsed laser beams were used to generate micro-bubbles on a CNT-PDMS composite film on which cells were adhered and cultured. Due to formation and collapse of bubbles in subseconds, cells can be detached in a non-thermal manner. This enables the harvested cells to be viable and cultured again for further studies, or lysed for molecular analysis (e.g., amplification, hybridization, sequencing, etc.). Combining the single cell capture scheme and the cell detachment and retrieval capability, one can monitor the development of a cell colony. With the reliable single cell capture scheme, each chamber starts with one single cell. Then, the clonal development of cells in each chamber is traced. It is possible to identify highly proliferative colony groups in a target chamber and detach all the cells in that chamber to compare the difference of the cells in that chamber against the whole population. In addition, in some embodiments, the daughter cells and the progenitor cells are harvested separately and the mRNA expression or other parameters are compared between the daughter cells and the progenitor cells. Such methods find use in the research, screening, and other therapeutic methods of regulation of cell differentiation (e.g., symmetric and asymmetric differentiation).

The methods and systems described herein result in high yield of cell retrieval without contamination of samples. Because a microfluidic platform is used, it is possible to easily retrieve the cells by controlling the flow. In the conventional dish-based technology it is difficult to control the flow for harvesting detached cells.

Experiments conducted during development of embodiments of the present disclosure resulted in high cell viability after detachment and re-growing of detached cells for further analysis. In addition, the systems and methods described herein have the advantage of the capability of tracing the development of cells from a single progenitor cell by clonal culture inside the same chamber, and detaching the target cell of interest at single cell resolution. Thus, the descendants of a single cell can be traced and studied.

The systems and methods described herein provide high spatial resolution of detaching cells down to a single-cell resolution. In some embodiments, partial detachment is utilized (e.g., to study the re-arrangement of the cytoskeleton after detachment).

Accordingly, embodiments of the present disclosure provide systems and methods for culturing and extracting single cells from a colony or growing cells. In some embodiments, the cells are growing or placed on a multilayer substrate. In some embodiments, the substrate comprises at least one energy (e.g., light or acoustic) absorbing layer (e.g., including but not limited to, carbon nanotube (CNT) film or a metal film) and at least one polymer layer.

The present disclosure is not limited to particular polymers for fabricating the polymer layer. Examples include, but are not limited to, polydimethylsiloxane (PDMS), polymethyl methacrylate (PMMA), or polystyrene (PS)). The polymer layer serves to both help cell adhesion and insulate cell from heat transferred from the metal layer to avoid cell damage in the detaching process.

In some embodiments, the energy (e.g., light or acoustic) absorbing layer is an acoustic absorbing layer that absorbs acoustic energy (e.g., sound waves) and generates heat.

In some embodiments, the light absorbing layer is a metal film. Examples of suitable metals include, but are not limited to, a gold film or a metal alloy film such as Au/Pd alloy or a composite of multiple metal layers.

In some embodiments, metal layers are patterned (e.g., into a regular pattern or shape). For example, in some embodiments, a mask or other barrier is used to restrict CVD of metal films to pre-defined areas of the substrate. In some embodiments, the patterns are used to restrict the area of detachment (e.g., cells will only detach from regions with metal layers). In some embodiments, metal layers are chemical vapor deposited and are thin (e.g., less than 10 μm). In some embodiments, polymer layers are approximately 1-100 μm thick. The metal layer absorbs heat from the energy source and transfer heat to the polymer layer.

In some embodiments, the light absorbing layer is a CNT. CNTs are produced by any suitable method, including, but not limited to, arc discharge, laser ablation, high-pressure carbon monoxide disproportionation, and chemical vapor deposition (CVD). Most of these processes take place in a vacuum or with process gases. CVD growth of CNTs can occur in vacuum or at atmospheric pressure. Large quantities of nanotubes can be synthesized by these methods.

In some embodiments, CNTs are generated by CVD. During CVD, a substrate is prepared with a layer of metal catalyst particles, most commonly nickel, cobalt, iron, or a combination (Inami, Nobuhito; Ambri Mohamed, Mohd; Shikoh, Eiji; Fujiwara, Akihiko (2007). “Synthesis-condition dependence of carbon nanotube growth by alcohol catalytic chemical vapor deposition method”. Sci. Technol. Adv. Mater. (PDF) 8 (4): 292; Ishigami; Ago, H; Imamoto, K; Tsuji, M; Iakoubovskii, K; Minami, N (2008). “Crystal Plane Dependent Growth of Aligned Single-Walled Carbon Nanotubes on Sapphire”. J. Am. Chem. Soc. 130 (30): 9918-9924). The metal nanoparticles can also be produced by other ways, including reduction of oxides or oxides solid solutions. The diameters of the nanotubes that are to be grown are related to the size of the metal particles. This can be controlled by patterned (or masked) deposition of the metal, annealing, or by plasma etching of a metal layer. The substrate is heated to approximately 700° C. To initiate the growth of nanotubes, two gases are bled into the reactor: a process gas (such as ammonia, nitrogen or hydrogen) and a carbon-containing gas (such as acetylene, ethylene, ethanol or methane). Nanotubes grow at the sites of the metal catalyst; the carbon-containing gas is broken apart at the surface of the catalyst particle, and the carbon is transported to the edges of the particle, where it forms the nanotubes. The catalyst particles can stay at the tips of the growing nanotube during growth, or remain at the nanotube base, depending on the adhesion between the catalyst particle and the substrate (Banerjee, Soumik, Naha, Sayangdev, and Ishwar K. Puri (2008). “Molecular simulation of the carbon nanotube growth mode during catalytic synthesis”. Applied Physics Letters 92 (23): 233121). Thermal catalytic decomposition of hydrocarbon is another option for the bulk production of CNTs. Fluidised bed reactor is the most widely used reactor for CNT preparation.

CVD is the most widely used method for the production of carbon nanotubes (Kumar, M. (2010). “Chemical vapor deposition of carbon nanotubes: a review on growth mechanism and mass production.”. Journal of Nanoscience and Nanotechnology 10: 6.). For this purpose, the metal nanoparticles are mixed with a catalyst support such as MgO or Al203 to increase the surface area for higher yield of the catalytic reaction of the carbon feedstock with the metal particles. One issue in this synthesis route is the removal of the catalyst support via an acid treatment, which sometimes could destroy the original structure of the carbon nanotubes. However, alternative catalyst supports that are soluble in water have proven effective for nanotube growth (Eftekhari, A.; Jafarkhani, P; Mortarzadeh, F (2006). “High-yield synthesis of carbon nanotubes using a water-soluble catalyst support in catalytic chemical vapor deposition”. Carbon 44 (7): 1343).

If a plasma is generated by the application of a strong electric field during growth (plasma-enhanced chemical vapor deposition), then the nanotube growth will follow the direction of the electric field (Ren, Z. F.; Huang, Z P; Xu, J W; Wang, J H; Bush, P; Siegal, M P; Provencio, P N (1998). “Synthesis of Large Arrays of Well-Aligned Carbon Nanotubes on Glass”. Science 282 (5391): 1105-7). By adjusting the geometry of the reactor it is possible to synthesize vertically aligned carbon nanotubes (e.g., perpendicular to the substrate). Without the plasma, the resulting nanotubes are often randomly oriented. Under certain reaction conditions, even in the absence of a plasma, closely spaced nanotubes will maintain a vertical growth direction resulting in a dense array of tubes resembling a carpet or forest.

The growth sites are controllable by careful deposition of the catalyst (Neupane, Suman; Lastres, Mauricio; Chiarella, M; Li, W. Z.; Su, Q; Du, G. H. (2012). “Synthesis and field emission properties of vertically aligned carbon nanotube arrays on copper”. Carbon 50 (7): 2641-50). In 2007, a team from Meijo University demonstrated a high-efficiency CVD technique for growing carbon nanotubes from camphor (Kumar, Mukul; Ando, Yoshinori (2007). “Carbon Nanotubes from Camphor: An Environment-Friendly Nanotechnology”. Journal of Physics: Conference Series 61: 643). Researchers at Rice University, until recently led by the late Richard Smalley, have concentrated upon finding methods to produce large, pure amounts of particular types of nanotubes. Their approach grows long fibers from many small seeds cut from a single nanotube; all of the resulting fibers were found to be of the same diameter as the original nanotube and are the same type as the original nanotube (Smalley, Richard E.; Li, Yubao; Moore, Valerie C.; Price, B. Katherine; Colorado, Ramon; Schmidt, Howard K.; Hauge, Robert H.; Barron, Andrew R.; Tour, James M. (2006). “Single Wall Carbon Nanotube Amplification: En Route to a Type-Specific Growth Mechanism”. Journal of the American Chemical Society 128 (49): 15824-15829).

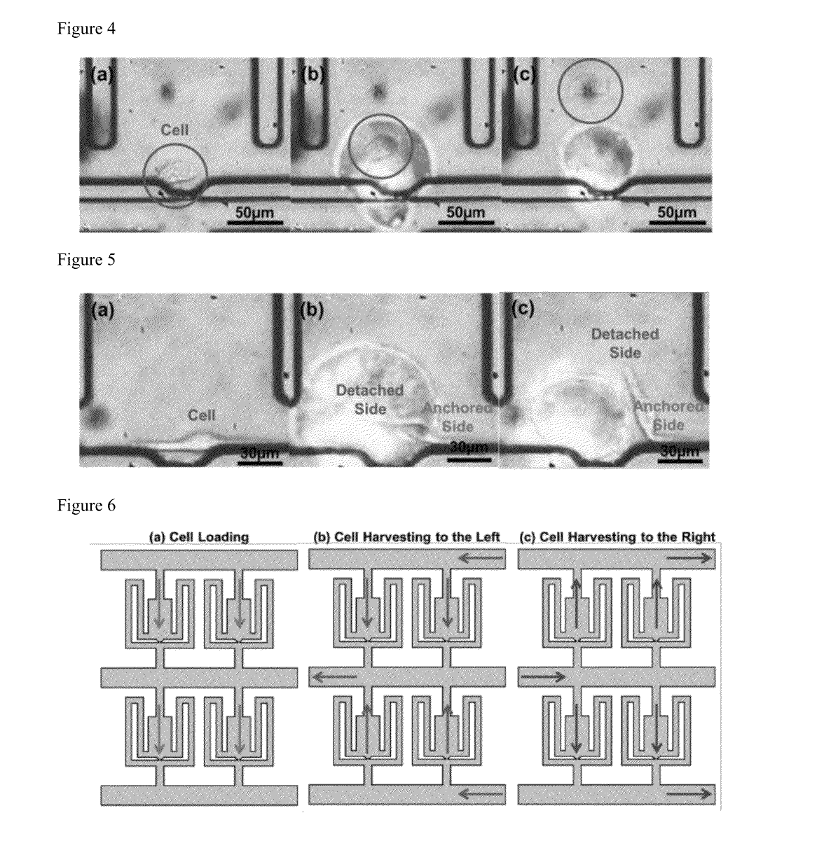

In some embodiments, the substrate comprises a plurality of bubbles between the layers. In some embodiments, the optical source (e.g., laser or pulsed laser (e.g., nanosecond pulsed laser) is used to generate micro bubbles. The bubbles form and collapse rapidly (e.g., within seconds) and allow cells to be detached from the substrate and/or cell colonies at a single cell resolution. In some embodiments, laser generated deformation of the substrate polymer leads to cell detachment. FIGS. 4, 5, and 7 illustrate detachment of single cells.

The present disclosure is not limited to a particular source of energy. Examples include optical, electrical, or acoustic. In some embodiments, the energy source is a laser. A laser is a device that emits light through a process of optical amplification based on the stimulated emission of electromagnetic radiation. In some embodiments, the laser is a pulsed laser. Pulsed operation of lasers refers to any laser not classified as continuous wave, so that the optical power appears in pulses of some duration at some repetition rate.

In some embodiments, lasers utilized in the systems and methods described herein are nanosecond pulsed lasers. The optical bandwidth of a pulse cannot be narrower than the reciprocal of the pulse width. In the case of extremely short pulses, that implies lasing over a considerable bandwidth, quite contrary to the very narrow bandwidths typical of CW lasers. The lasing medium in some dye lasers and vibronic solid-state lasers produces optical gain over a wide bandwidth, making a laser possible which can thus generate pulses of light as short as a few femtoseconds (10-15 s).

In some embodiments, the substrates are integrated into a microfluidic device. The combination of cell growth and detachment substrate and microfluidic device provides the advantages of being able to study the difference between two daughter cells (e.g., from the same colony) and the ability to retrieve cells after detachment and easily transfer retrieved cells to a second cell growth/detachment chamber. FIG. 6 illustrates the ability to retrieve cells in multiple directions via microfluidic channels for transfer to new chamber. In addition, retrieved cells are viable and can be further cultured in new chambers (e.g., for downstream analysis).

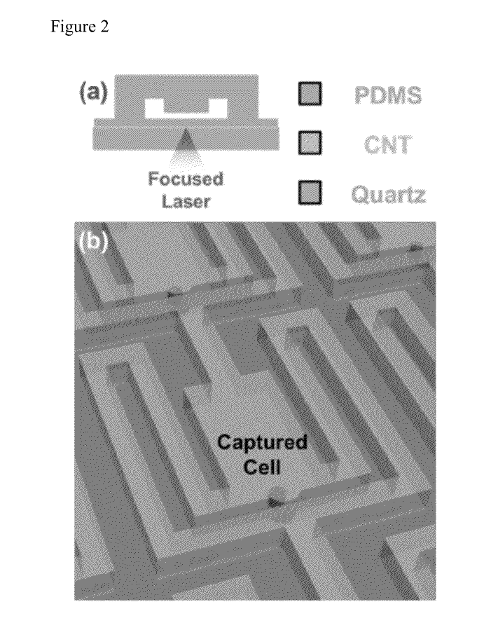

In some embodiments, the microfluidic device comprises a plurality of chambers (e.g., each configured to hold a single cell or cell colony) as shown in FIG. 2. In some embodiments, each well is approximately 400 μm by 400 μm. In some embodiments, devices comprise one or more arrays of 64 cells in an 8 by 8 arrangement, although numbers of wells are specifically contemplated (e.g., 100 or more, 1000 or more, etc.). This array is approximately 5 mm by 6 mm. In some embodiments, the microfluidic devices comprise fluid control components to allow for exchange or culture medium or other agents (e.g., test compounds). In some embodiments, the microfluidic device comprises a plurality of channels (See e.g., FIG. 6). The channels allow for removal and segregation of single detached cells. In some embodiments, detached cells are placed in new chambers for further growth or are removed for molecular analysis.

The present disclosure is not limited to particular methods for fabricating microfluidic devices. In some embodiments, devices are made from poly-dimethylsiloxane (PDMS).

In some embodiments, layers are made by supplying a negative “master” and casting a castable material over the master. Castable materials include, but are not limited to, polymers, including epoxy resins, curable polyurethane elastomers, polymer solutions (e.g., solutions of acrylate polymers in methylene chloride or other solvents), curable polyorganosiloxanes, and polyorganosiloxanes which predominately bear methyl groups (e.g., polydimethylsiloxanes (“PDMS”)). Curable PDMS polymers are well known and available from many sources. Both addition curable and condensation-curable systems are available, as also are peroxide-cured systems. All of these PDMS polymers have a small proportion of reactive groups which react to form crosslinks and/or cause chain extension during cure. Both one part (RTV-1) and two part (RTV-2) systems are available. Additional curable systems are preferred when biological particle viability is needed.

In some embodiments, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers are well known. See, e.g. D. C. Duffy et al., “Rapid Prototyping of Microfluidic Systems in Poly(dimethylsiloxane),” Analytical Chemistry 70, 4974-4984 (1998). See also, J. R. Anderson et al., Analytical Chemistry 72, 3158-64 (2000); and M. A. Unger et al., Science 288, 113-16 (2000), each of which is herein incorporated by reference in its entirety.

In some embodiments, fluids are supplied to the device by any suitable method. Fluids may, for example, be supplied from syringes, from microtubing attached to or bonded to the inlet channels, etc.

Fluid flow may be established by any suitable method. For example, external micropumps suitable for pumping small quantities of liquids are available. Micropumps may also be provided in the device itself, driven by thermal gradients, magnetic and/or electric fields, applied pressure, etc. All these devices are known to the skilled artisan. Integration of passively-driven pumping systems and microfluidic channels has been proposed by B. H. Weigl et al., Proceedings of MicroTAS 2000, Enshede, Netherlands, pp. 299-302 (2000).

In other embodiments, fluid flow is established by a gravity flow pump, by capillary action, or by combinations of these methods. A simple gravity flow pump consists of a fluid reservoir either external or internal to the device, which contains fluid at a higher level (with respect to gravity) than the respective device outlet. Such gravity pumps have the deficiency that the hydrostatic head, and hence the flow rate, varies as the height of liquid in the reservoir drops. For many devices, a relatively constant and non-pulsing flow is desired.

To obtain constant flow, a gravity-driven pump as disclosed in published PCT application No. WO 03/008102 A1 (Jan. 18, 2002), herein incorporated by reference, may be used. In such devices, a horizontal reservoir is used in which the fluid moves horizontally, being prevented from collapsing vertically in the reservoir by surface tension and capillary forces between the liquid and reservoir walls. Since the height of liquid remains constant, there is no variation in the hydrostatic head.

Flow may also be induced by capillary action. In such a case, fluid in the respective outlet channel or reservoir will exhibit greater capillary forces with respect to its channel or reservoir walls as compared to the capillary forces in the associated device. This difference in capillary force may be brought about by several methods. For example, the walls of the outlet and inlet channels or reservoirs may have differing hydrophobicity or hydrophilicity. Alternatively, the cross-sectional area of the outlet channel or reservoir is made smaller, thus exhibiting greater capillary force.

In some embodiments, flow is facilitated by embedded capacitor valves that pump fluids in a separate channel when pressurized. This is achieved by having a series of valves in the bottom that direct a pressurized gas or liquid causing the membrane to deform and squeeze the fluid in the top channel forward. Additional control is provided by having valves in the top layer that can open sequentially.

### II. Methods

Embodiments of the present disclosure provide methods of detaching single cells from colonies and performing downstream analysis. The present disclosure is not limited to a particular cell type. The systems and methods described herein find use with a variety of cell types, including prokaryotic and eukaryotic cells and single cell organisms. In some embodiment, human or mammal cells are utilized (e.g., primary cells, stem cells (e.g., cancer stem cells), immortalized cells, cancer cell lines, etc.).

In some embodiments, cells are grown and separated using the systems described herein. In some embodiments, separated cells are placed in a separate chamber of the device and further cultured or grown. In some embodiments, molecular properties of parent and daughter cells or different cell types from the same organ or tumor are analyzed and compared. In some embodiments, live cells are analyzed. In some embodiments, intact fixed cells are analyzed. In some embodiments, cells are lysed and molecular analysis is performed.

The present disclosure is not limited to particular types of analyses. Examples include, but are not limited to, screening cells for gene expression at the mRNA or protein level (e.g., via reporter genes in live cells or molecular analysis); screening compounds (e.g., drugs) for their effect on cell growth, cell death, viral infectivity, or gene expression; screening viruses for infectivity (e.g., plaque formation); epigenome analysis (e.g., methylation status of genes and/or promoters), protein analysis (e.g., immunoassays such as e.g., single cell Western blot and mass spectrometry analysis), copy-number variations (CNVs) assays, and screening for mutations or polymorphisms (e.g., SNPs).

The present disclosure is not limited to particular analysis methods. Examples include, but are not limited to, sequencing analysis, hybridization analysis, and amplification analysis. Exemplary analysis methods are described herein.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., those described in table 1) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The methylation levels of non-amplified or amplified nucleic acids can be detected by any conventional means. For example, in some embodiments, Methylplex-Next Generation Sequencing (M-NGS) methodology is utilized. In other embodiments, the methods described in U.S. Pat. Nos. 7,611,869, 7,553,627, 7,399,614, and/or 7,794,939, each of which is herein incorporated by reference in its entirety, are utilized. Additional detection methods include, but are not limited to, bisulfate modification followed by any number of detection methods (e.g., probe binding, sequencing, amplification, mass spectrometry, antibody binding, etc.) methylation-sensitive restriction enzymes and physical separation by methylated DNA-binding proteins or antibodies against methylated DNA (See e.g., Levenson, Expert Rev Mol Diagn. 2010 May; 10(4): 481-488; herein incorporated by reference in its entirety).

In some embodiments, gene expression or other protein analysis (e.g., detection of cell surface antigens) is performed using immunoassays or mass spectrometry.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

### EXAMPLES

The following examples are provided in order to demonstrate and further illustrate certain preferred embodiments and aspects of the present invention and are not to be construed as limiting the scope thereof.

### Example 1

### Cell Detachment Mechanism and Fabrication of Microfluidic Platform

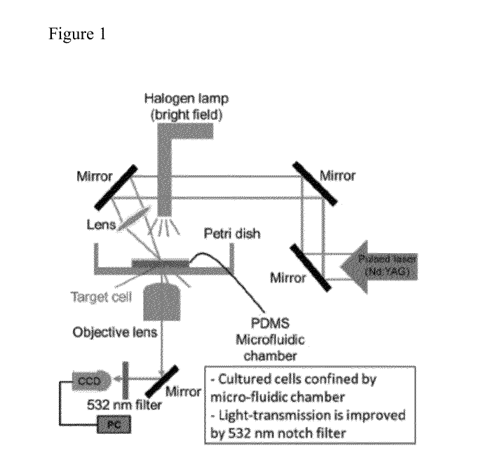

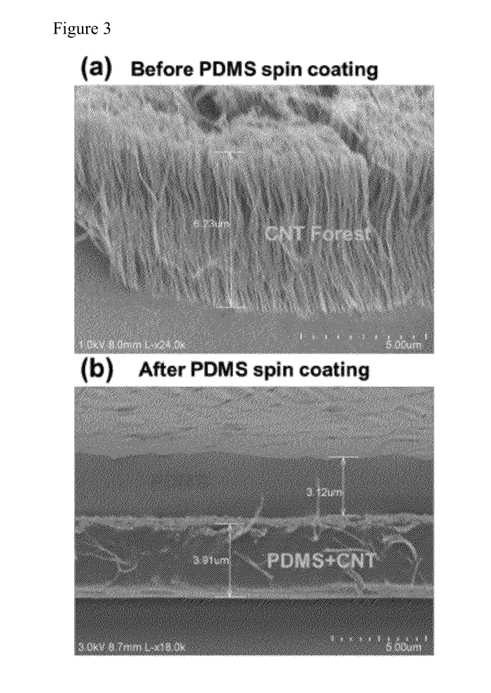

An exemplary cell detachment technology system is illustrated in the FIG. 1. To absorb the optical power and transform it to mechanical force for cell detachment, a two-layer substrate (shown in FIG. 2(a)) composed of a light absorbing material and a polymer layer was developed. The light absorbing layer transforms the optical energy to heat, and the generated heat leads to the expansion of a bubble trapped in PDMS. The quick (<1 μs) deformation of PDMS leads to a high shear stress, which detached the cells. Due to the high optical absorption characteristics of CNTs, a CVD (Chemical Vapor Deposited) CNT film was first investigated as a light absorbing layer. The SEM picture of the CNT film (˜6 μm) grown on the substrate is shown in the FIG. 3, before and after coating of a polymer (PDMS) layer. It was also demonstrated that a thin (10-200 nm) Au/Pd alloy film was be used as an alternative for a light absorbing layer.

Using a thin polymer (PDMS) layer (˜3 μm) on top of the CNT film provides two benefits: 1) high thermal expansion and the trapped bubble in the PDMS helps transformation of the heat to a mechanical shock wave (Baac et al., (2012) Sci. Rep. 2, 989), and 2) low thermal conductance that isolates the heat from the cells above, so that the generated heat does not affect cell viability. The thickness of PDMS layers should be optimized to effective cell detachment with minimum optical power. It was found that the thickness should be in the range of 1-10 μm, although other ranges are contemplated. The PDMS was spun on the CNT film at a spinning speed of 6,000 rpm after diluting it in a solvent (Hexane or toluene).

### Selective Cell Detachment and Retrieval at Single Cell Resolution

In the platform, a hydrodynamic capture scheme was used to capture cells at single cell resolution in each chamber with high efficiency (Chung et al., Appl. Phys. Lett, 98(12), 3701 (2011)). A schematic of the platform is shown in FIG. 2(b).) A single cell capture rate of −80-90% was obtained in the fabricated platform. After capturing cells, cell assays (e.g., including, but not limited to, drug screening, cell-to-cell interaction, cell migration, sphere formation, cell differentiation, etc. can be performed) In this platform, cells, including primary cells, were cultured with high viability for more than 14 days. After identifying the target cell of interest to be harvested for further analysis such as genotyping, a short-pulse of laser (6 ns) is applied for cell detachment at single cell resolution. The laser energy (0.1 mJ) is absorbed by the CNT layer grown on the substrate (FIG. 3). Under laser irradiation, micro-bubbles are formed, inducing high shear force to detach the cell. Low thermal conductivity of the PDMS layer makes an ideal (thermal) insulating layer, so the generated heat does not affect cell viability. FIG. 4 illustrates the detaching process of a single skov3 (ovarian cancer) cell. FIG. 5 demonstrates spatial control over detachment. After focusing the irradiation to the one side of the cell, the cell was partially detached such that it dangled only on the anchored opposite side. After detachment, cells are harvested from the device for further analysis including, for example, mRNA PCR followed by gene sequencing.

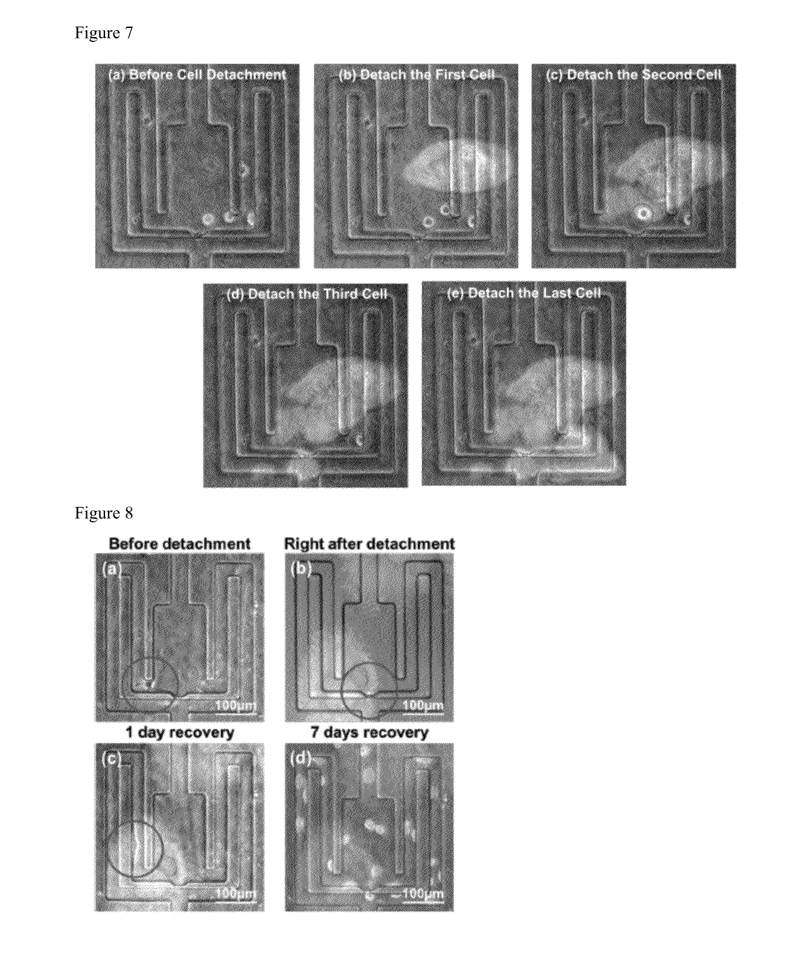

The cell retrieval device described herein achieves a high yield and avoids undesired contamination from residual cells left in the inlet. In the cell loading phase, media flows from inlet to outlet (in FIG. 6, flowing from top to bottom), so the cells are captured at the capture site (FIG. 6(a)). In the detachment phase, the cells are first detached from the lower chambers (the second row) by applying pressure from the right (FIG. 6(b)). The detached cells in the lower chambers are guided upward and retrieved in the left outlet. Then, the upper chambers (the first row) are detached by applying pressure from the left (FIG. 6(c)). The detached cells in the upper chambers are guided upward and retrieved in the right outlet. Using alternating parallel channels in an array, all the cells from the left and right inlets, respectively, were retrieved so the residual cells during cell loading did not contaminate the sample. FIG. 7 demonstrates the sequential detachment process of four cells from a chamber.

### Viability of Detached Cells

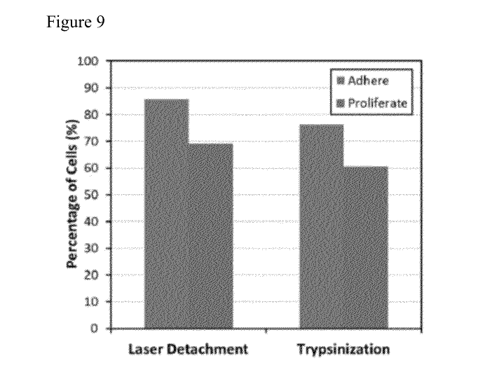

FIG. 8 shows cell viability after recovered from cell detachment. One day after detachment, the cell re-adhered to the substrate, and then it proliferated to twenty-five cells after seven days. FIG. 9 shows a comparison of cell viability with the conventional trypsinization-based method. The cell detachment method described herein showed a better cell viability than the conventional method. These data demonstrated that the single cell detachment/retrieval methods described herein provide extremely important capability in single cell analysis and cancer biology for clinical applications of drug development and personalized medicine by understanding cell behaviors and correlating them with genotypic signatures for heterogeneous cell populations and their state transitions.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, in vitro fertilization, development, or related fields are intended to be within the scope of the following claims.

1. A system, comprising:
a) a multi-layer substrate comprising a light absorbing layer and a polymer layer; and
b) a source of optical energy.

2. The system of claim 1, wherein said substrate comprises a plurality of bubbles.

3. The system of claim 1, wherein said polymer layer is selected from Polydimethylsiloxane (PDMS), polymethyl methacrylate (PMMA), and polystyrene (PS).

4. The system of claim 1, wherein said polymer layer is 1-100 μm thick.

5. The system of claim 1, wherein said light absorbing layer is selected from a carbon nanotube film and a metal film.

6. The system of claim 1, wherein said light absorbing layer is chemical vapor deposited.

7. The system of claim 1, wherein said light absorbing layer is patterned.

8. The system of claim 1, wherein said polymer layer is on top of said light absorbing layer.

9. The system of claim 1, wherein said polymer film has a plurality of cells attached thereof.

10. The system of claim 9, wherein said cells are growing.

11. The system of claim 9, wherein contacting said substrate with said optical source generates heat that leads to the expansion of said bubbles or deformation of said substrate and detachment of cells growing on said bubbles or substrate.

12. The system of claim 11, wherein said system further comprises a plurality of reagents and devices for performing analysis of said cells.

13. The system of claim 1, wherein said source of optical energy is a nanosecond pulsed laser.

14. The system of claim 1, wherein said substrate is a component of a microfluidic chamber.

15. The system of claim 14, wherein said microfluidic chamber comprises a fluid exchange system.

16. The system of claim 15, wherein said microfluidic chamber comprises a plurality of chambers, wherein each of said chambers is configured to hold a single cell or cell colony.

17. A method, comprising:
contacting a multi-layer substrate comprising a light absorbing layer and a polymer layer wherein said polymer film has a plurality of cells attached thereof with a source of optical energy such that one or more single cells are detached from said substrate to generate detached cells and attached cells.

18. The method of claim 17, further comprising the step of performing molecular analysis on said detached cell and/or said attached cells.

19. The method of claim 31, wherein said molecular analysis is selected from gene expression analysis, epigenomic analysis, gene copy number variation analysis, protein analysis, sequencing analysis, and mutation analysis.
